(19) United States
(12) Patent Application Publication
(10) Pub. No.: US 2016/0325053 A1
(43) Pub. Date: Nov. 10, 2016

(54) Needle Disposal Unit

(71) Applicant: Antonio Sagnotti, Cary, NC (US)

(72) Inventor: Antonio Sagnotti, Fairview, NJ (US)

(21) Appl. No.: 15/210,982

(22) Filed: Jul. 15, 2016

Related U.S. Application Data
(63) Continuation-in-part of application No. 14/465,046, filed on Aug. 21, 2014, now abandoned.
Application No. 14/465,046 is a continuation-in-part of application No. 12/602,351, filed on Nov. 30, 2009, now Pat. No. 8,154,593.
Application No. 12/602,351 is a continuation-in-part of application No. 12/378,839, filed on Feb. 20, 2009, now abandoned.

(51) Int. Cl. A61M 5/32; H05B 1/02

(52) U.S. Cl. CPC A61M 5/3278; H05B 1/025; A61M 2005/3283

(57) ABSTRACT

A portable needle destroying device for safely destroying a needle while attached to a syringe housing. The needle destroying device includes a housing, an opening in the housing to receive a needle and syringe housing, and a flexible cover to substantially cover the opening in the housing. The device further includes a surface fixed within the interior of the housing and away from the opening, the surface in electrical resistive capacity, a battery in electrical communication with the surface, and a motion sensor proximate to the opening and in communication with the battery. When a needle is inserted through the opening, the motion sensor activates the battery which heats the surface, the needle contacts the surface and melts upon contact.

The present application is a continuation-in-part of pending U.S. patent application Ser. No. 14/465,046, filed Aug. 21, 2014, entitled “Needle Disposal Unit”, which is a continuation-in-part of U.S. patent application Ser. No. 12/602,351, filed Sep. 4, 2012, entitled “Needle Disposal Unit” which is a continuation-in-part of U.S. patent application Ser. No. 12/378,839, filed on Feb. 20, 2009, entitled “Needle Disposal Unit” which claims the benefit of priority of provisional patent application Ser. No. 61/066,717, filed on Feb. 21, 2008, entitled “Needle Disposal Unit”, all of which are incorporated herein in their entirety.

### BACKGROUND

1. Field

The embodiments described herein relate generally to a needle disposal unit and, more particularly, to an efficient, portable needle disposal unit designed to prevent any spark formation.

2. Description of the Prior Art

Physicians, nurses, MASH units, and other health-care workers are always subject to the danger of infection from the patients they treat. Airborne pathogens like the viruses which cause influenza or the common cold come immediately to mind, and are as likely to infect health-care workers as they are schoolteachers; but in this age of HIV/AIDS, the dangers of viral transmission from patient to health-care worker are not merely inconvenient, they can be lethal.

The primary route of exposure to blood borne pathogens is accidental percutaneous injury caused by needle sticks (puncturing of the skin by a needle). In the United States, approximately 800,000 needle stick injuries occur in hospitals annually. Studies have shown that many of these injuries occur after a sharp needle or other sharp object is used with as many as one-third of all needle injuries have been reported to occur during disposal activities.

To counter the dangers associated with these viral infections transmitted by needles, i.e.,—hypodermic needles, syringes, and lancets, the health-care industry has adopted the use of units designed for the safe collection of needles. These containers, when filled, are generally destroyed by compaction or incineration. Still, disposing of hypodermic needles and syringes is inherently dangerous, even with existing collection systems. But, while health authorities concede that no needle collection or disposal system is perfect; it does make sense that disposing of the needles immediately rather than first collecting them and then disposing of them at some subsequent point, is preferable.

In addition to the health risks posed by needles, the disposal of needles by means of melting has its own risks. Any metal that is heated may spark which creates a risk in a medical environment where gas, namely oxygen is used frequently. To date, there are no portable needle disposal mechanisms designed to minimize the risk of spark formation while effectively and efficiently disposing of needles.

### SUMMARY

The embodiments herein are directed to a needle destroying device for safely destroying a needle while attached to a syringe housing. The needle destroying device includes a housing, an opening in the housing to receive a needle and syringe housing, and a cover to substantially cover the opening in the housing. The device further includes a battery and a surface in electrical communication with the battery and heated by the battery.

In an alternative embodiment, the device further includes a motion sensor to activate the battery and heat the surface.

A further embodiment herein described is directed to a portable needle destroying device for safely destroying a needle while attached to a syringe housing. The needle destroying device includes a housing, an opening in the housing to receive a needle and syringe housing, and a flexible cover to substantially cover the opening in the housing. The device also includes a surface fixed within the interior of the housing and away from the opening. The surface functioning in an electrical resistive capacity. The device yet further includes a battery in electrical communication with the surface and a motion sensor proximate to the opening and in communication with the battery. When a needle is inserted through the opening, the motion sensor activates the battery which heats the surface, the needle contacts the surface and melts upon contact.

Another embodiment is directed to a method for safely destroying a needle while the needle is attached to a syringe housing. The method includes the steps of providing a needle destroying device having a housing, an opening in the housing to receive a needle and syringe, a cover to substantially cover the opening in the housing, a battery and a surface in electrical communication with the battery. The method also includes the steps of activating the battery, inserting a needle attached to a syringe into the needle opening, and contacting the needle to the surface, thereby destroying the needle.

### BRIEF DESCRIPTION OF THE DRAWINGS

FIG. 1 is a perspective view of the first embodiment described herein;

FIG. 2 is a sectional view of the embodiment of FIG. 1;

FIG. 3 is a front view of the embodiment of FIG. 1;

FIG. 4 is a rear view of the embodiment of FIG. 1; and

FIG. 5 is a diagrammatic representation of the electrical arrangement of the embodiment of FIG. 1.

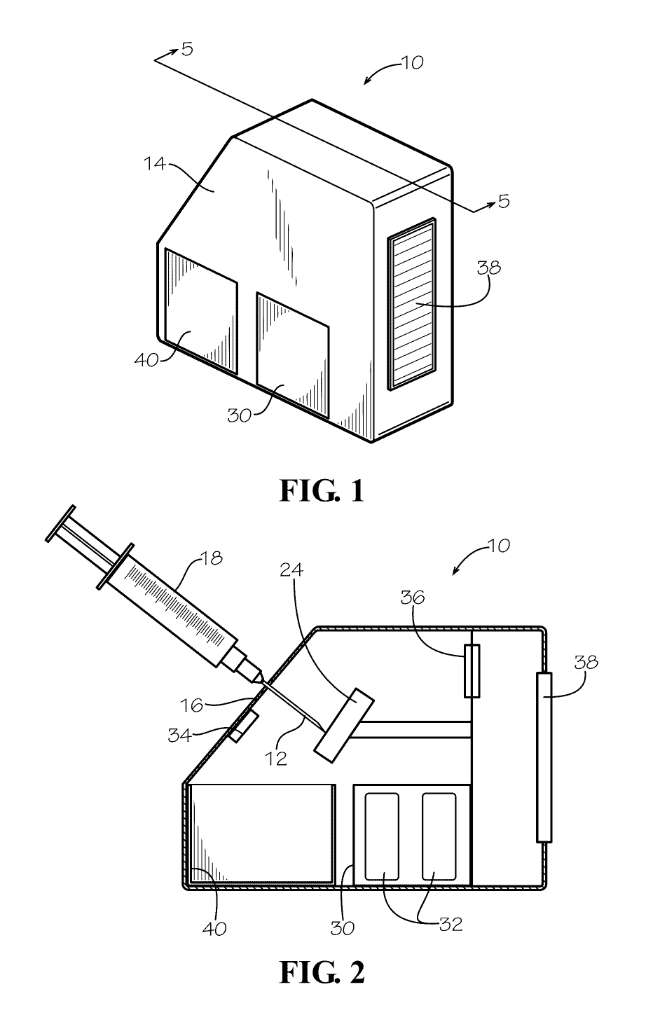

### DETAILED DESCRIPTION OF THE PREFERRED EMBODIMENTS

The preferred embodiment 10 is directed to a needle disposal unit, providing health-care workers a safe and efficient means of destroying used needles 12. Preferably, the first preferred embodiment includes a rectangular housing 14, approximately five (5″) inches in height by approximately five (5″) inches in width by approximately six (6″) inches. The housing 14 is preferably made of PEEK (polyether ether keytone). PEEK is lightweight, strong and highly resistive to heat.

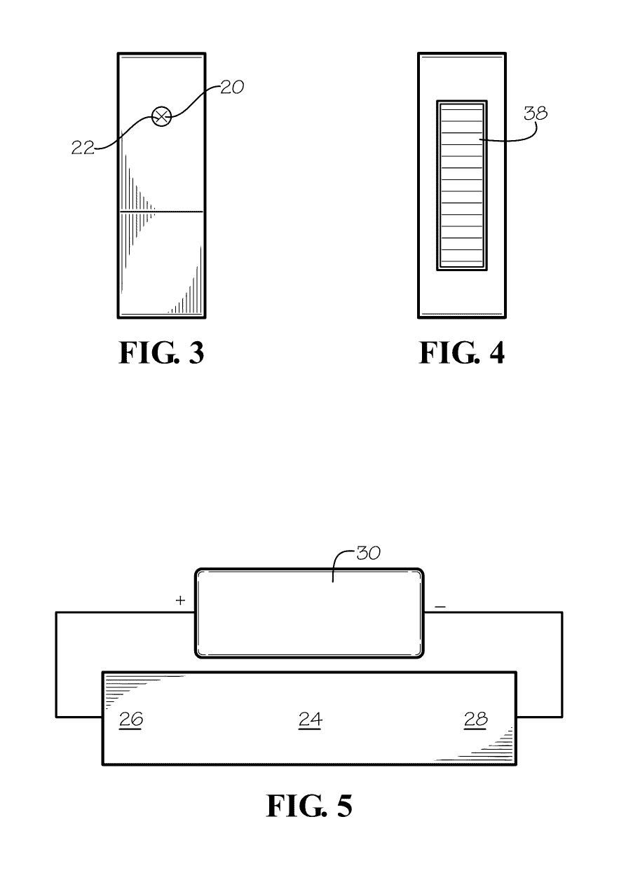

Turning now to FIG. 2, the embodiment 10 includes an opening 16 which is sized to accommodate medical grade needles 12 and corresponding syringes 18. The opening 16 is covered on the exterior of the housing 14 with a flexible cover 20, as shown in detail in FIG. 3. The cover 20 is preferably made of a neoprene material and has a slit 22 in the middle in the form of an “X”. The slit 22 acts as a flap to shield the syringe 18 during operation which will be addressed in more detail below.

Returning to FIG. 2, the carbon block 24 is fixed to the interior of the housing 14. The carbon block 24 is preferably rectangular in shape and located opposite to the opening 16. It is preferred to have the carbon block 24 located as far away from the opening 16 as possible to avoid sparking which will be discussed in more detail below.

The carbon block 24 has a first end 26 and a second end 28. The embodiment further includes a battery tray 30. The battery tray 30 includes a pair of 6 volt rechargeable batteries 32 arranged in parallel within the tray in such a way that enables the batteries 32 to be removed from the housing by sliding the tray 30 out of the housing and replacing the tray with a new tray having recharged batteries. Alternatively, the batteries 32 themselves may be charged separately and replaced within the tray.

The first end 26 of the carbon block 24 is in electrical communication with the battery tray 30, as shown in FIG. 5. Preferably this is accomplished with a wire connecting the first end 26 of the carbon block 24 to the battery tray 30 with a wire (not shown). The second end 28 of the carbon block 24 is also in electrical communication with the battery tray 30. In this arrangement, the carbon block is acting in a resistive capacity. When the battery is activated, electrical energy is provided to both the first 26 and second 28 ends of the carbon block. The electrical energy heats up the carbon block. Using a pair of 6 volt batteries 32, it is anticipated that the carbon block would heat to at least 2500° F. In addition, it is anticipated that the time in which to achieve a temperature of at least 2500° F. is about 6 seconds.

A motion sensor 34 is fixed to the housing 14 near the opening 16, as shown in FIG. 2. The motion sensor 34 is in electrical communication with the batteries 32. When activated, the motion sensor 34 activates the batteries 32 and causes them to heat up.

Also, within the first embodiment 10 is a safety exhaust-fan 36, shown in FIG. 2 and an air-filter 38, shown in FIG. 4, designed to provide effective filtration and odor-control. The safety exhaust fan 36 and air filter are also in electrical communication with the battery pack 30, and the motion sensor 34. Preferably, the air filter 38 is an electronic air filter that collects particles by electrically charging the air. The air is pulled, again typically by a fan, past a series of oppositely charged metal plates (not shown). The charged particles are attracted to the plates and removed from the airstream. The collection plates only need to be replaced periodically and occasionally wiped clean.

In use, a user inserts a used needle 12 attached to a syringe 18 through the opening 16 and cover 20. As the length of the needle 12 is inserted into the opening 16 it activates the motion sensor 34. The activation of the motion sensor 34 causes the batteries 32 to activate. This causes electrical energy to move to the carbon block 24 which acts as a resistor and heats up. The carbon block 24 will heat to at least 2500° F. in a matter of 6 seconds.

As the needle 12 continues to travel into the interior of the housing 14 it encounters the carbon block 24 heated surface. When the needle 12 contacts the heated surface of the carbon block 24 it begins to melt. As the user continues to apply pressure to the needle 12, the length of the needle melts as it contacts the heated carbon block 24. When the entire length of the needle 12 has melted, the tip of the syringe 18 is melted shut and any remaining liquid within the syringe is sealed within the syringe. At this time, the user, sensing the end of needle travel, removes the syringe from the opening and disposes of the sealed syringe accordingly.

When a needle is introduced into the opening 20, the motion sensor also activates the exhaust fan 36 and air filter 38. The exhaust fan 36 pulls air out of the interior of the housing 14 and causes it to pass through the air filter 38. This cleans the air within the interior before it is released to the housing exterior.

It is anticipated that during the heating and subsequent melting of the needle 12 onto the carbon block 24, a spark may form. The cover 20 physically shields the exterior environment from any spark formation once the needle 12 makes contact with the carbon block. The cover 24 and flaps formed by the slit 22 provide maximum protection to the exterior from any potential minor combustion occurring within the housing 14 interior.

The battery arrangement of two 6 volt batteries arranged in parallel enables a user to destroy approximately 190 needles before requiring the batteries to be recharged. After approximately 400 uses, the needle disposal unit 10 is emptied by opening the disposal door ½″, holding the unit over a trash-can or needle container, and gently tilting the needle disposal unit 10 until the door faces downward and the fragments fall out.

Beyond maintaining a charged battery and wiping the first embodiment 10 off with a damp cloth from time to time, the unit requires only the emptying of needle fragments every 190 uses.

The first embodiment 10 is a compact and portable unit for destroying used hypodermic needles 12 and rendering used syringes 18 harmless and presents a number of distinct and significant benefits and advantages. Foremost, because the first embodiment 10 does not require the user to detach the used hypodermic needle 12 from the syringe 14, the potential of an accidental needle stick is greatly diminished. The user merely inserts the needle 12 into the needle disposal unit 12, employing only one hand to hold the syringe 14, and never touching the needle 12, and the first embodiment 10 effectively destroys the needle 12. Further, the first embodiment 10, by sterilizing the lowermost portion, or “hub”, of the syringe 14, effectively ruins the syringe 14 at the same time it cauterizes or sterilizes it. The user is then left with a safely neutralized syringe 14, which can then be deposited with complete safety into a standard “Sharps” container.

The foregoing exemplary descriptions and the illustrative preferred embodiments of the present invention have been explained in the drawings and described in detail, with varying modifications and alternative embodiments being taught. While the invention has been so shown, described and illustrated, it should be understood by those skilled in the art that equivalent changes in form and detail may be made therein without departing from the true spirit and scope of the invention, and that the scope of the present invention is to be limited only to the claims except as precluded by the prior art. Moreover, the invention as disclosed herein may be suitably practiced in the absence of the specific elements which are disclosed herein. Further the terms “interior” and “exterior” are for reference purposes only and are in no way intended to limit the scope of the embodiments described herein.

What is claimed is:

1. A needle destroying device for safely destroying a needle while attached to a syringe housing, the needle destroying device comprising:
a housing;
an opening in the housing to receive a needle and syringe housing;
a cover to substantially cover the opening in the housing;
a battery; and
a surface in electrical communication with the battery and heated by the battery.

2. The needle destroying device of claim 1 further comprising a motion sensor to activate the battery and heat the surface.

3. The needle destroying device of claim 1 wherein the surface is made of carbon.

4. The needle destroying device of claim 1 wherein the surface is planar.

5. The needle destroying device of claim 1 wherein the surface is heated to at least 2500° F.

6. The needle destroying device of claim 5 wherein the surface is heated within 6 seconds.

7. The needle destroying device of claim 1 wherein the housing is made of PEEK.

8. The needle destroying device of claim 1 wherein the battery is 12 volts.

9. A method for safely destroying a needle while the needle is attached to a syringe housing, the method comprising the steps of:
providing a needle destroying device having a housing, an opening in the housing to receive a needle and syringe, a cover to substantially cover the opening in the housing, a battery and a surface in electrical communication with the battery;
activating the battery so as to cause the surface to heat up;
inserting a needle attached to a syringe into the needle opening; and
causing the needle to contact the surface, thereby destroying the needle.

10. The method of claim 9 further comprising the step of providing a motion sensor proximate to the opening and in communication with the battery whereby when the needle is inserted into the opening, the battery is activated to heat the surface.

11. The method of claim 9 wherein the surface is planar.

12. The method of claim 9 wherein the battery is rechargeable.

13. The method of claim 9 wherein when the surface is heated to a temperature of at 2500° F.

14. The method of claim 13 wherein the surface is heated within 6 seconds.

15. The method of claim 9 wherein the housing is made of PEEK.

16. A portable needle destroying device for safely destroying a needle while attached to a syringe housing, the needle destroying device comprising:
a housing;
an opening in the housing to receive a needle and syringe housing;
a flexible cover to substantially cover the opening in the housing;
a surface fixed within the interior of the housing and away from the opening, the surface in electrical resistive capacity;
a battery in electrical communication with the surface; and
a motion sensor proximate to the opening and in communication with the battery, whereby when a needle is inserted through the opening, the motion sensor activates the battery which heats the surface, the needle contacts the surface and melts upon contact.

17. The portable needle destroying device of claim 16 wherein the surface is made of carbon.

18. The portable needle destroying device of claim 16 wherein the housing is made of PEEK.

19. The portable needle destroying device of claim 16 wherein the surface heats up to a temperature of at least 2500° F.

20. The portable needle destroying device of claim 19 wherein the surface heats up within 6 seconds.
